(19) United States
(12) Patent Application Publication
(10) Pub. No.: US 2024/0084383 A1
(43) Pub. Date: Mar. 14, 2024

(54) METHODS FOR SPATIAL ANALYSIS USING ROLLING CIRCLE AMPLIFICATION AND DETECTION PROBES

(71) Applicant: 10x Genomics, Inc., Pleasanton, CA (US)

(72) Inventors: Eswar Prasad Ramachandran Iyer, Oakland, CA (US); Yifeng Yin, Elk Grove, CA (US); Sultan Doganay Tuncer, Danville, CA (US)

(21) Appl. No.: 18/511,481

(22) Filed: Nov. 16, 2023

Related U.S. Application Data
(63) Continuation of application No. 17/531,093, filed on Nov. 19, 2021, now Pat. No. 11,827,935.
(60) Provisional application No. 63/115,916, filed on Nov. 19, 2020.

(51) Int. Cl. C12Q 1/6874; C12Q 1/6855; C12Q 1/6862

(52) U.S. Cl. CPC C12Q 1/6874; C12Q 1/6855; C12Q 1/6862

(57) ABSTRACT

Provided herein are methods of improving sensitivity of spatial detection of an analyte in a biological sample using splint oligonucleotides, circularized second strands, and rolling circle amplification. For example, provided herein are methods of improving sensitivity of spatial detection of an analyte in a biological sample where a splint oligonucleotide hybridizes to a second strand; the second strand is ligated together thereby creating a circularized second strand, rolling circle amplification of the circularized second strand results in generation of an amplified second strand, and all or part of the sequence of the amplified second strand is determined and used to spatially detect the analyte in the biological sample.

### CROSS-REFERENCE TO RELATED APPLICATIONS

This application is a continuation of U.S. application Ser. No. 17/531,093, filed on Nov. 19, 2021, which claims the benefit of U.S. Provisional Application Ser. No. 63/115,916, filed Nov. 19, 2020, the entire contents of which are incorporated by reference herein.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Generally, spatial analysis requires determining the sequence of the analyte sequence or a complement thereof and the sequence of the spatial barcode or a complement thereof in order to identify spatial location of the analyte. Typically, this requires sequencing which can be time and resource intensive. Therefore, there is a need to assess analyte and biological sample quality prior to spatial analysis.

### SUMMARY

Spatial analysis requires processing of a captured analyte in order to determine its abundance and location in a biological sample. During this process, after capture of the analyte, a process of second strand synthesis is performed. This process includes generating a single-stranded nucleic acid that is complementary both to the capture probe and to the analyte (or a complement thereof). After, the second strand can be further processed, placed in a library, and sequenced. The disclosure has identified that sensitivity can be lost during second strand synthesis. To address this issue, the disclosure provides methods of generating an amplified product comprising the capture probe or complement thereof and analyte or complement thereof using a splint oligonucleotide and rolling circle amplification—all performed on a substrate.

In one embodiment, disclosed herein is a method of determining location and abundance of an analyte in a biological sample. In some instances, the method includes: (a) hybridizing the analyte to a capture probe on an array, wherein the capture probe comprises a spatial barcode and a capture domain; (b) extending the capture probe using the analyte as a template, thereby generating an extended capture probe, and generating a second strand comprising a sequence that is complementary to (i) the analyte or a complement thereof and (ii) the spatial barcode or a complement thereof; (c) denaturing the second strand from the extended capture probe under conditions wherein a 5′ end of the second strand and a 3′ end of the second strand dehybridize from the extended capture probe; (d) hybridizing a splint oligonucleotide both to the 5′ end of the second strand and to the 3′ end of the second strand; (e) generating a circularized second strand; (f) amplifying the circularized second strand, thereby creating an amplified second strand; and (g) determining all or part of the sequence of the amplified second strand to determining the location and the abundance of the analyte in the biological sample.

In some instances, the analyte comprises a capture domain capture sequence that hybridizes to the capture domain, and wherein the capture domain comprises a poly(T) sequence. In some instances, the capture domain capture sequence comprises a poly(A) sequence. In some instances, the array comprises a plurality of capture probes.

In some instances, extending the capture probe and generating a second strand utilize a polymerase or reverse transcriptase. In some instances, denaturing comprises increasing the temperature, thereby dehybridizing the 5′ end of the second strand and the 3′ end of the second strand from the extended capture probe. In some instances, before step (e) above, the method includes extending the 5′ end of the second strand using the splint oligonucleotide as a template, thereby creating an extended 5′ end. In some instances, generating the circularized second strand comprises ligating the extended second portion to the first portion using a ligase. In some instances, the ligase is a T4 DNA ligase.

In some instances, amplifying the circularized second strand comprises rolling circle amplification (RCA) using the circularized second strand as a template. In some instances, the amplified second strand comprises (i) the spatial barcode or complement thereof and (ii) all or part of the analyte or a complement thereof. In some instances, the method further includes hybridizing an oligonucleotide to a portion of the amplified second strand, thereby producing a double-stranded sequence, and wherein the double-stranded sequence comprises a restriction site. In some instances, the method further includes digesting the double-stranded sequence using a restriction enzyme.

In some instances, the splint oligonucleotide comprises a first sequence that is substantially complementary to the 5′ end of the second strand, and a second sequence that is substantially complementary to the 3′ end of the second strand. In some instances, the capture probe further comprises one or more functional domains, a unique molecular identifier, a cleavage domain, and combinations thereof. In some instances, the method further includes phosphorylating the 5′ end of the splint oligonucleotide prior to the ligation step; and/or phosphorylating the 5′ end of the first portion of the second strand. In some instances, the amplifying step comprises hybridizing one or more amplification primers to the circularized second strand, and amplifying the circularized second strand with a polymerase.

In some instances, the determining step comprises sequencing all or part of the sequence of the amplified second strand to determining the location and the abundance of the analyte in the biological sample.

In some instances, the biological sample comprises a formalin-fixed, paraffin-embedded (FFPE) tissue sample, a fresh tissue sample, or a frozen tissue sample.

Also disclosed herein are kits. In some instances, the kits include (a) an array comprising a plurality of capture probes, wherein a capture probe of the plurality of capture probes comprises (i) a capture domain that hybridizes to an analyte of a biological sample and (ii) a spatial barcode; (b) one or more splint oligonucleotides and a ligase; (c) one or more RCA primers and a Phi29 DNA polymerase; (d) one or more restriction enzymes; and (e) instructions for performing any of the methods disclosed herein.

Also disclosed herein is a method of spatially detecting of an analyte in a biological sample comprising: (a) hybridizing the analyte to a capture probe comprising a spatial barcode and creating a second strand comprising a sequence that is complementary to a portion of the analyte and an extended capture probe; (b) denaturing the second strand under conditions wherein a first portion of the second strand and a second portion of the second strand de-hybridize from the extended capture probe; (c) hybridizing a splint oligonucleotide to the first portion and to the second portion; (d) ligating part of the splint oligonucleotide, to the first portion, and the second portion thereby creating a circularized second strand; (e) amplifying the circularized second strand, thereby creating an amplified second strand; and (f) determining all or part of the sequence of the amplified second strand to spatially detect the analyte in the biological sample.

Also disclosed herein is a method of spatially detecting of an analyte in a biological sample comprising: (a) hybridizing the analyte to a capture probe comprising a spatial barcode and creating a second strand comprising a sequence that is complementary to a portion of the analyte and an extended capture probe; (b) denaturing the second strand under conditions wherein a first portion of the second strand and a second portion of the second strand de-hybridize from the extended capture probe; (c) hybridizing a splint oligonucleotide to the first portion and to the second portion; (d) ligating part of the splint oligonucleotide, to the first portion, and the second portion thereby creating a circularized second strand; (e) amplifying the circularized second strand, thereby creating an amplified second strand; and (f) determining all or part of the sequence of the amplified second strand to spatially detect the analyte in the biological sample.

In some instances, the capture probe is on a substrate comprising a plurality of capture probes, wherein the capture probe comprises a capture domain and the spatial barcode, and wherein the analyte comprises a capture probe binding domain that is capable of binding to the capture domain. In some instances, the splint oligonucleotide comprises a backbone sequence, wherein the backbone sequence comprises a double-stranded sequence. In some instances, the methods further include extending the second portion using the splint oligonucleotide as a template, thereby creating an extended second portion. In some instances, the methods further include ligating the extended second portion to the first portion, thereby creating a circularized second strand. In some instances, the methods further include extending the capture probe, thereby creating an extended capture probe. In some instances, the methods further include amplifying the extended capture probe creating a second strand comprising a sequence that is complementary to a portion of the analyte and the extended capture probe. In some instances, the determined sequence of the amplified second strand comprises the spatial barcode or complementary sequence thereof for spatially detecting the analyte in the biological sample. In some instances, the splint oligonucleotide comprises a first sequence that is substantially complementary to the second portion, and a second sequence that is substantially complementary to the first portion. In some instances, the methods further include digesting the amplified second strand. In some instances, the method improves sensitivity of spatial detection of an analyte as compared to methods of spatially detecting an analyte that does not include rolling circle amplification.

Also disclosed herein is a method of spatially detecting of an analyte in a biological sample comprising: (a) hybridizing the analyte to a capture probe comprising a spatial barcode and extending the capture probe, thereby creating an extended capture probe; (b) amplifying the extended capture probe creating a second strand comprising a sequence that is complementary to a portion of the analyte and the extended capture probe; (c) hybridizing a splint oligonucleotide to a first portion of the second strand and a second portion of the second strand, (d) ligating part of the splint oligonucleotide, or a complement thereof, to the first portion and the second portion creating a circularized second strand; (e) amplifying the circularized second strand creating an amplified second strand and digesting the amplified second strand, thereby producing a plurality of second strand fragments; and (f) determining all or part of the sequence of a second strand fragment, and using the determined sequence to spatially detect the analyte in the biological sample. In some instances, the capture probe is on a substrate comprising a plurality of capture probes, wherein the capture probe comprises a capture domain and the spatial barcode, and wherein the analyte comprises a capture probe binding domain that is capable of binding to the capture domain. In some instances, the splint oligonucleotide comprises a backbone sequence, wherein the backbone sequence comprises a double-stranded sequence. In some instances, the methods further include denaturing the second strand under conditions wherein a first portion of the second strand and a second portion of the second strand de-hybridize from the extended capture probe. In some instances, the splint oligonucleotide comprises a first sequence that is substantially complementary to the second portion, and a second sequence that is substantially complementary to the first portion. In some instances, the determined sequence of the second strand fragment comprises the spatial barcode of the capture probe or a complementary sequence thereof.

In some instances, digesting comprises inducing a plurality of double-stranded breaks in the amplified second strand, wherein a double-stranded break occurs at a specific nucleic acid position(s) in the amplified second strand. In some instances, digesting comprises: hybridizing an oligonucleotide to a portion of the amplified second strand, thereby producing a double-stranded sequence, wherein the double-stranded sequence comprises a restriction site; and digesting the double-stranded sequence using a restriction enzyme. In some instances, the restriction site is added to the circularized second strand during the amplifying step. In some instances, the amplifying step comprises rolling circle amplification using the circularized second strand as a template. In some instances, a second strand fragment of the plurality of second strand fragments comprises all or part of the analyte or complement thereof and a spatial barcode or a complement thereof.

In some instances, the methods further include amplifying the second strand fragment prior to determining the sequence of the second strand fragment, thereby generating an amplified second strand fragment. In some instances, the methods further include determining the sequence of the amplified second strand fragment, wherein the determined sequence of the amplified second strand fragment comprises the spatial barcode sequence of the capture probe or a complementary sequence thereof, and using the determined sequence of the amplified second strand fragment or the spatial barcode to spatially detect the analyte in the biological sample. In some instances, the amplifying and digesting steps are performed concurrently.

In some instances, the oligonucleotide comprises a blocking moiety on the 3′ end.

In some instances, the method improves sensitivity of spatial detection of an analyte as compared to methods of spatially detecting an analyte that do not include any method disclosed herein.

In some instances, the second portion of the second strand comprises one or more of a spatial barcode, a unique molecular identifier, and a primer sequence. In some instances, the second portion of the second strand comprises a portion of the captured analyte. In some instances, the first portion of the second strand comprises a primer sequence. In some instances, the first portion of the second strand comprises a portion of the captured analyte. In some instances, the splint oligonucleotide further comprises one or more of a functional sequence and a unique barcode. In some instances, the functional sequence is a primer sequence. In some instances, the primer is used for amplifying the circularized second strand.

In some instances, the ligating step comprises a T4 DNA ligase.

In some instances, the methods further include phosphorylating the 5′ end of the double-stranded splint backbone sequence of the splint oligonucleotide prior to the ligation step. In some instances, the methods further include phosphorylating the 5′ end of the first portion of the second strand.

In some instances, the capture domain comprises a sequence that is at least partially complementary to the analyte. In some instances, the capture domain of the capture probe comprises a homopolymeric sequence. In some instances, the capture domain of the capture probe comprises a poly(T) sequence. In some instances, the capture domain of the capture probe comprises a non-homopolymeric sequence. In some instances, the non-homopolymeric sequence is a random sequence, a partially random sequence or a fully defined sequence. In some instances, the capture probe comprises a cleavage domain. In some instances, the cleavage domain comprises a cleavable linker selected from a photocleavable linker, a UV-cleavable linker, an enzyme-cleavable linker, or a pH-sensitive cleavable linker. In some instances, extending the capture probe comprises reverse transcribing the analyte or complementary sequence thereof. In some instances, extending the capture probe comprises generating a sequence that is complementary to a portion of the analyte. In some instances, reverse transcribing the analyte generates a reverse complement of a template switching oligonucleotide. In some instances, amplifying the extended capture probe comprises annealing a template switching oligonucleotide primer to the reverse complement of the template switching oligonucleotide. In some instances, amplifying further comprises hybridizing the 3′ end of the extended capture probe to the first portion of the second strand, and using the 3′ end of the extended capture probe as a substrate in a rolling circle amplification reaction.

In some instances, the amplifying step (h) comprises: hybridizing one or more amplification primers to the circularized second strand, the capture probe, or the analyte; and amplifying the circularized second strand with a polymerase. In some instances, the determining step comprises sequencing. In some instances, the sequencing comprises generating a sequencing library of the amplified second strand, second strand fragments, or amplified second strand fragments.

In some instances, the biological sample comprises a FFPE sample. In some instances, the biological sample comprises a tissue section. In some instances, the biological sample comprises a fresh frozen sample. In some instances, the biological sample comprises live cells.

Also disclosed herein is a composition comprising the circularized second strand, wherein the circularized second strand comprises a first portion of the second strand, a portion of the second strand that remained hybridized to the extended capture probe, a second portion of the second strand, and a portion of the backbone sequence of the splint oligonucleotide.

Also disclosed herein is a kit, comprising: a substrate for spatial detection of an analyte, one or more splint oligonucleotides and a ligase; one or more RCA primers and a Phi29 DNA polymerase; and instructions for performing any of the methods disclosed herein.

Also disclosed herein is a kit, comprising: a substrate for spatial detection of an analyte; one or more splint oligonucleotides and a ligase; one or more RCA primers and a Phi29 DNA polymerase; one or more oligonucleotides and one or more restriction enzymes; and instructions for performing any of the methods disclosed herein.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating exemplary, non-limiting, non-exhaustive steps for second strand synthesis.

FIG. 3A is a schematic illustrating exemplary, non-limiting, non-exhaustive steps for generating a circularized second strand.

FIG. 3B is a schematic illustrating exemplary, non-limiting, non-exhaustive steps for amplifying a circularized second strand.

FIG. 4 is a schematic illustrating exemplary, non-limiting, non-exhaustive steps for digesting an amplified second strand using oligonucleotides, partially double stranded amplified second strands, and a restriction enzyme.

### DETAILED DESCRIPTION

Disclosed herein are methods of improving sensitivity of spatial detection of an analyte in a biological sample. The techniques disclosed herein facilitate downstream processing by increasing the abundance of an analyte (e.g., increasing copies of an analyte or derivatives thereof) from a biological sample. For example, an analyte or an analyte derived molecule (e.g., a second strand cDNA molecule) is amplified prior to determining all or part of the sequence of the analyte. The amplification method described herein amplifies a spatial barcode of a capture probe or a complement thereof in addition to all or part of the sequence of an analyte or a complement thereof. This enables retention of spatial information. A splint oligonucleotide is hybridized to a first portion and a second portion of a second strand (e.g., a second strand cDNA molecule), where the second strand includes the spatial barcode or a complement thereof and all or part of the sequence of the analyte or a complement thereof. In some cases, hybridization of the splint oligonucleotide to the first portion and the second portion is enabled by denaturing the second strand hybridized to the extended capture probe under conditions that allow the first portion and the second portion to de-hybridize from the extended capture probe. The splint oligonucleotide mediates ligation of the second portion to the first portion of the second strand thereby producing a circularized second strand. The circularized second strand is amplified thereby creating an amplified second stand that includes the spatial information necessary (e.g., information including the sequence the analyte or a complement thereof and the sequence of spatial barcode or a complement thereof) to spatially detect the analyte in the biological sample. “Improving sensitivity” of spatial detection as used herein refers to an increased detection of an analyte at a location of a sample using methods disclosed herein compared to a reference sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

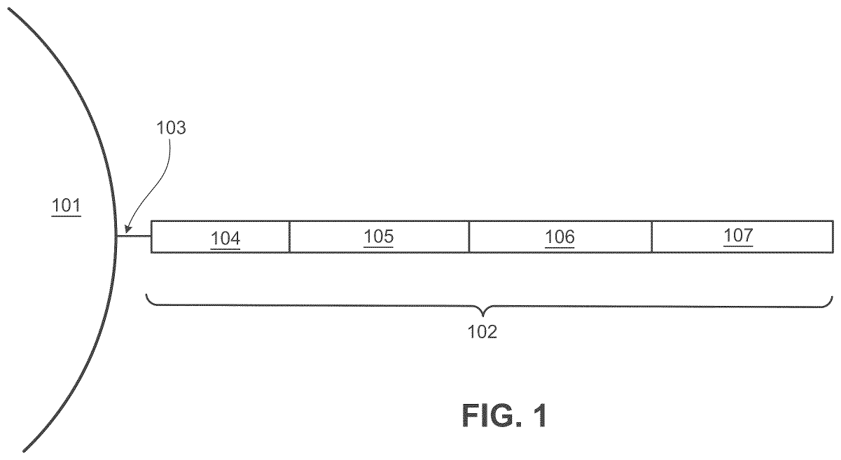

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or part of sequencer specific flow cell attachment sequence (e.g., a P5 or P7 sequence), all or part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Surface Amplification on a Substrate to Improve Sensitivity of Spatial Detection

This disclosure features methods of improving sensitivity of spatial detection of an analyte in a biological sample. In other methods of spatial analysis, after capture of an analyte on a capture probe affixed to an array, a second strand, which is complementary to the capture probe and to all or part of the analyte, is generated, followed by cDNA amplification, library preparation of the amplified secondary strand, and sequencing. The methods provided herein disclose an alternative method of spatial detection. In part, disclosed herein are methods using a splint oligonucleotide that can bind to a second strand (e.g., a second strand cDNA molecule bound to an analyte that is bound to a capture probe) and enable ligation of the second portion to the first portion thereby creating a circularized second strand. The circularized second strand can be amplified, all or part of the sequence of the amplified second strand can be determined, and the determined sequence used to spatially detect the analyte in the biological sample. This disclosure also features methods of improving sensitivity of spatial detection of an analyte in a biological sample using fragmentation (e.g., digestion) of the amplified second strand. In a non-limiting example, the method includes digesting the amplified second strand thereby generating second strand fragments, and determining all or part of the sequence of the second strand fragments to spatially detect the analyte in the biological sample.

This disclosure also features a method of improving sensitivity of spatial detection of an analyte in a biological sample where the method includes: providing a biological sample including an analyte on a substrate, wherein the substrate includes a plurality of capture probes comprising capture domains that are capable of hybridizing to an analyte sequence; hybridizing the analyte to the capture domain and creating a second strand including a sequence that is complementary to a portion of the analyte and the extended capture probe; hybridizing a splint oligonucleotide to a first portion of the second strand and to a second portion of the second strand; ligating part of the splint oligonucleotide, to the first portion, and the second portion thereby creating a circularized second strand; amplifying the circularized second strand, thereby creating an amplified second strand; and determining all or part of the sequence of the amplified second strand to spatially locate the analyte in the biological sample. In some embodiments, the method further includes extending the capture probe creating an extended capture probe. In some embodiments, the method further includes amplifying the extended capture probe creating a second strand including a sequence that is complementary to a portion of the analyte and the extended capture probe. In some embodiments, the method further includes subjecting the second strand to a denaturing step under conditions wherein the first portion of the second strand and the second portion of the second strand de-hybridize from the extended capture probe.

This disclosure also features a method of improving sensitivity of spatial detection of an analyte in a biological sample where the method includes: a biological sample including an analyte on a substrate wherein the substrate includes a plurality of capture probes comprising a capture domain, and wherein the analyte includes a capture probe binding domain that is capable of binding to the capture domain; hybridizing the capture probe binding domain to the capture domain and creating a second strand including a sequence that is complementary to a portion of the analyte and the extended capture probe; denaturing the second strand under conditions wherein a first portion of the second strand and a second portion of the second strand de-hybridize from the extended capture probe; hybridizing a splint oligonucleotide to the first portion and to the second portion; ligating part of the splint oligonucleotide, to the first portion, and the second portion thereby creating a circularized second strand; amplifying the circularized second strand, thereby creating an amplified second strand; and determining all or part of the sequence of the amplified second strand to spatially detect the analyte in the biological sample. In some embodiments, the method further includes extending the capture probe creating an extended capture probe. In some embodiments, the method further includes amplifying the extended capture probe creating a second strand including a sequence that is complementary to a portion of the analyte and the extended capture probe.

In some embodiments, the method further includes digesting the amplified second strand. In some embodiments, the amplifying step and the digesting step are performed in one reaction. For example, the method can include a step concurrently amplifying the circularized second strand creating an amplified second strand and digesting the amplified second strand, thereby producing a plurality of second strand fragments.

In a non-limiting example, this disclosure features a method improving sensitivity of spatial detection of an analyte in a biological sample where the method includes: a biological sample including an analyte on a substrate, wherein the substrate includes a plurality of capture probes comprising a capture domain, and wherein the analyte includes a capture probe binding domain that is capable of binding to the capture domain; hybridizing the capture probe binding domain to the capture domain and extending the capture probe creating an extended capture probe; amplifying the extended capture probe creating a second strand including a sequence that is complementary to a portion of the analyte and the extended capture probe; hybridizing a splint oligonucleotide to a first portion of the second strand and to a second portion of the second strand, ligating part of the splint oligonucleotide, or a complement thereof, to the first portion and the second portion creating a circularized second strand; concurrently amplifying the circularized second strand creating an amplified second strand and digesting the amplified second strand, thereby producing a plurality of second strand fragments; and determining all or part of the sequence of a second strand fragment, and using the determined sequence to spatially detect the analyte in the biological sample. In some embodiments, the method further includes subjecting the second strand to a denaturing step under conditions wherein a first portion of the second strand and a second portion of the second strand de-hybridize from the extended capture probe.

(a) Splint Oligonucleotide and Circularization of the Second Strand

This disclosure features methods for improving sensitivity of spatial detection of an analyte in a biological sample using a splint oligonucleotide hybridized to a second strand. In some embodiments, the method includes ligating part of the splint oligonucleotide to a first portion of the second strand and to a second portion of the second strand, thereby creating a circularized second strand. As used herein, a “splint oligonucleotide” refers to an oligonucleotide that has, at its 5′ and 3′ ends, sequences (e.g., a first sequence at the 5′ end and a second sequence at the 3′ end) that are complementary to portions (e.g., a first portion and a second portion) of the second strand. Upon hybridization to the first and second portions of the second strand, the splint oligonucleotide brings the two portions of the second strand into contact, allowing circularization of the second strand by ligation (e.g., ligation using any of the methods described herein). Upon hybridization to the first and second portions of the second strand, the second portion of the second strand is extended until the two portions of the second strand are brought into contact, allowing circularization of the second strand by ligation (e.g., ligation using any of the methods described herein). The ligation product can be referred to as the “circularized second strand.” In some embodiments, after circularization of the second strand, rolling circle amplification can be used to amplify the circularized second strand creating an “amplified second strand.”

In some embodiments, a first sequence of a splint oligonucleotide includes a sequence that is substantially complementary to a second portion of the second strand. In some embodiments, the second portion of the second strand is 3′ to the first portion of the second strand. In some embodiments, the first sequence is at least 70% identical (e.g., at least 75% identical, at least 80% identical, at least 85% identical, at least 90% identical, at least 95% identical, or at least 99% identical) to the second portion.

In some embodiments, the splint oligonucleotide includes a backbone sequence. In some embodiments, the backbone sequence is double stranded while the first sequence and second sequences are single stranded. In some embodiments, the splint oligonucleotide is double stranded. In some embodiments, the backbone sequence includes a sequence that is substantially complementary to an amplification primer. The amplification primer can be a primer used in a rolling circle amplification reaction (RCA), where the RCA increases the “copy number” of the analyte and analyte derived molecules. In some embodiments, the backbone sequence includes a functional sequence. In some embodiments, the backbone sequence includes a restriction site. For example, the backbone sequence includes a sequence that is recognized by a restriction enzyme. In such cases, the backbone sequence can be converted from a single stranded sequence to a double stranded sequence (e.g., a double stranded sequence that includes a functional restriction site) by adding an oligonucleotide that is substantially complementary or by performing a nucleic acid extension reaction. In some embodiments, a double stranded backbone sequence includes a restriction site. In some instances, the restriction site is not in the captured analyte. In some instances, the restriction site is not in the genome of the biological sample.

In some embodiments, a second sequence of a splint oligonucleotide includes a sequence that is substantially complementary to a first portion of the second strand. In some embodiments, the first portion of the second strand is 3′ to the second portion of the second strand. In some embodiments, the second sequence is at least 70% identical (e.g., at least 75% identical, at least 80% identical, at least 85% identical, at least 90% identical, at least 95% identical, or at least 99% identical) to the first portion.

In some embodiments, the splint oligonucleotide does not include a backbone sequence. In such cases, the first sequence and the second sequence are directly adjacent to each other on the splint oligonucleotide. As such, when the splint oligonucleotide is hybridized to the second strand, the first portion of the second strand and the second portion of the second strand hybridize to adjacent sequences on the splint oligonucleotide. This enables ligation without having to perform a gap filling step.

In some embodiments, the splint oligonucleotide includes a backbone sequence where the first sequence is not directly adjacent to the second sequence. In such cases, a “gap” exists on the splint oligonucleotide between where the first sequence is hybridized to the second portion and where the second sequence is hybridized to the first portion. In some embodiments, the splint oligonucleotide includes a sequence (e.g., a gap) between the first sequence and the second sequence of at least 1-100, 1-90, 1-80, 1-70, 1-60, 1-50, 1-40, 1-30, 1-20, 1-10, 1-9, 1-8, 1-7, 1-6, 1-5, 1-4, 1-3, 1-2 or 1 nucleotide(s). In a non-limiting example, a first sequence having a sequence that is substantially complementary to a second portion of the second strand and a second sequence having a sequence that is substantially complementary to a first portion of the second strand each bind to the second strand leaving a sequence (e.g., the “gap”) in between the first and second sequences that is gap-filled thereby enabling ligation and generation of the circularized second strand. In some instances, to generate a splint oligonucleotide that includes a first sequence and a second sequence that are close enough to one another to initiate a ligation step, the second sequence is extended enzymatically (e.g., using a reverse transcriptase).

In some embodiments, the “gap” sequence between the first sequence and the second sequence include one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides.

In some embodiments, the gap is filled by extending the second portion of the second strand. In some embodiments, extending the second portion of the second strand includes a nucleic acid extension reaction (e.g., any of the nucleic acid extension reactions described herein). In some embodiments, extending the second portion of the second strand includes reverse transcribing the splint oligonucleotide. In some embodiments, extending the second portion of the second strand includes using a reverse transcriptase (e.g., any of the reverse transcriptases described herein). In some embodiments, extending the second portion of the second strand includes using a Moloney Murine Leukemia Virus (M-MulV) reverse transcriptase. In some embodiments, the reverse transcriptase includes strand displacement properties. In some embodiments, extending the second portion of the second strand generates a sequence that is complementary to the splint oligonucleotide. In some embodiments, extending the second portion of the second strand generates an extended second sequence of the second strand that is complementary to the splint oligonucleotide. In some embodiments, extending the second portion of the second strand generates a sequence that is adjacent to the first portion of the second strand.

In some embodiments, the ligation step includes ligating the second portion to the first portion of the second strand using enzymatic or chemical ligation. In some embodiments where the ligation is enzymatic, the ligase is selected from a T4 RNA ligase (Rnl2), a SplintR ligase, a single stranded DNA ligase, or a T4 DNA ligase. In some embodiments, the ligase is a T4 RNA ligase (Rnl2) ligase. In some embodiments, the ligase is a pre-activated T4 DNA ligase as described herein. A non-limiting example describing methods of generating and using pre-activated T4 DNA include U.S. Pat. No. 8,790,873, the entire contents of which are herein incorporated by reference.

(b) Amplification of Circularized Second Strand

This disclosure features methods for improving sensitivity of spatial detection of an analyte in a biological sample by amplifying the circularized second strand. In a non-limiting example, the method includes an amplifying step wherein one or more amplification primers are hybridized to the circularized second strand and the circularized second strand is amplified using a polymerase. In another non-limiting example, the method includes an amplifying step where a 3′ end of the extended capture probe is used as a primer and the circularized second strand is amplified using a polymerase. In some embodiments, the amplifying step increases the copy number of the second strand. The sequence of the amplified second strand can then be determined and used to spatially detect the analyte in the biological sample. In some embodiments, the amplifying step includes rolling circle amplification (RCA).

As used herein, rolling circle amplification (RCA) can refer to a polymerization reaction carried out using a single-stranded circular DNA (e.g., a circularized second strand) as a template and an amplification primer that is substantially complementary to the single-stranded circular DNA (e.g., the circularized second strand) to synthesize multiple continuous single-stranded copies of the template DNA (e.g., the circularized second strand). In some embodiments, RCA includes hybridizing one or more amplification primers to the circularized second strand and amplifying the circularized strand using a Phi29 DNA polymerase. In addition, RCA can refer to a polymerization reaction carried out using a single-stranded circular DNA (e.g., a circularized second strand) as a template and the 3′ end of the extended capture probe as a primer to synthesize multiple continuous single-stranded copies of the template DNA (e.g., the circularized second strand). In such cases, the 3′ end of the extended capture is substantially complementary to a portion (e.g., a portion corresponding to the first portion of the second strand) of the single-stranded circular DNA (e.g., the circularized second strand). For example, RCA can include hybridizing the 3′ end of the extended capture probe to the circularized second strand and amplifying the circularized strand using a Phi29 DNA polymerase.

In some embodiments, an amplification primer includes a sequence that is substantially complementary to the first portion or the second portion of the circularized second strand. For example, the amplification primer can be substantially complementary to the first portion or a complement thereof. By substantially complementary, it is meant that the amplification primer is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in the circularized second strand.

In some embodiments, the amplifying step includes hybridizing the 3′ end of the extended capture probe to the first portion of the second strand, and using the 3′ end of the extended capture probe as a substrate in a rolling circle amplification (RCA) reaction. In such cases, the method includes a denaturing step under conditions where the 3′ end of the extended capture probe is de-hybridized from the first portion of the second strand. During the subsequent processing of the second strand (e.g., ligation and circularization) the second strand can remain hybridized to the extended capture probe. Therefore, the 3′ end of the extended capture probe can be re-hybridized to the first portion of the second strand.

In some embodiments, non-limiting examples of DNA polymerase include: B su DNA polymerase, Bst DNA polymerase, VENT™ DNA polymerase, DEEPVENT™ DNA polymerase, Phi29 DNA polymerase, Klenow fragment, T4 DNA polymerase and T7 DNA polymerase enzymes. In some embodiments, the DNA polymerase is Phi29 DNA polymerase. In some embodiments, the term “DNA polymerase” includes not only naturally-occurring enzymes but also all modified derivatives thereof, including derivatives of naturally-occurring DNA polymerase enzymes. For instance, in some embodiments, the DNA polymerase is modified to remove 5′-3′ exonuclease activity. Sequence-modified derivatives or mutants of DNA polymerase enzymes that can be used include, but are not limited to, mutants that retain at least some of the functional, e.g., DNA polymerase activity of the wild-type sequence.

(c) Capture Probe Extension, Second Strand Synthesis and De-Hybridizing

This disclosure features methods for improving sensitivity of spatial detection of an analyte in a biological sample using de-hybridization of a second strand, hybridization of a splint oligonucleotide, circularization of the second strand, and amplification of a circularized second strand. In a non-limiting example, the method includes capturing an analyte, extending a capture probe, generating a second strand, de-hybridizing a first and second portion of the second strand, hybridizing a splint oligonucleotide to the second strand, circularizing the second strand and amplifying the circularized second strand.

In some embodiments, extending the capture probe includes performing a nucleic acid extension reaction. In some embodiments, extending the capture probe includes reverse transcribing the analyte or complementary sequence thereof. In some embodiments, extending the capture probe includes generating a sequence that is complementary to a portion of the analyte. In some embodiments, extending the capture probe includes attaching a template switching oligonucleotide to the analyte. In some embodiments, reverse transcribing the analyte generates a reverse complement of a template switching oligonucleotide.

In some embodiments, amplifying the extended capture probe includes annealing a template switching oligonucleotide (TSO) primer to a reverse complement of the template switching oligonucleotide (rcTSO). In some embodiments, extending the capture probe and/or amplifying the extended capture probe includes using a polymerase. A non-limiting example of a polymerase is a DNA polymerase.

In some embodiments, extending the capture probe includes a reverse transcriptase enzyme, where the enzyme includes one or more of terminal transferase activity, template switching ability, strand displacement ability, or combinations thereof. In some embodiments, the terminal transferase activity of the reverse transcriptase adds untemplated nucleotides to the 3′ end of the cDNA molecule. In some embodiments, the reverse transcriptase adds 1, 2, 3, 4, 5, 6, 7, 8, 9, 10, or more untemplated nucleotides to the 3′ end of the cDNA molecule. In some embodiments the first enzyme includes a Moloney Murine Leukemia Virus (M-MLV) reverse transcriptase enzyme. In some embodiments the first enzyme includes a Moloney Murine Leukemia Virus (M-MLV) reverse transcriptase enzyme and the second enzyme is a Bst DNA polymerase. In some embodiments the first enzyme includes a Moloney Murine Leukemia Virus (M-MLV) reverse transcriptase enzyme and the second enzyme is a Phi29 DNA polymerase.

In some embodiments, second strand synthesis is performed by a DNA polymerase selected from the group including, but not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes. In some embodiments, the second strand synthesis is a Phi29 DNA polymerase. In some embodiments, the second strand synthesis is performed by a Bst DNA polymerase.

In some embodiments, the method includes contacting the analyte bound to the capture probe with a composition that includes a template switching oligonucleotide (TSO). In some embodiments, the TSO includes an untemplated nucleotide region and a TSO primer region. In some embodiments, the length of a template switching oligonucleotide can be at least about 1, 2, 10, 20, or 50 nucleotides or longer. In some embodiments, the length of a template switching oligonucleotide can be at most about 2, 10, 20, 50, 100, 150, 200, or 250 nucleotides or longer.

In some embodiments, the TSO primer region includes a sequence that is at least partially complementary to the TSO primer. In some embodiments, the TSO primer region includes a sequence that is 1, 2, 3, 4, 5, 6, 7, 8, 9, 10, 11, 12, 13, 14, 15, 16, 17, 18, 19, 20, 21, 22, 23, 24, 25, or nucleotides in length. In some embodiments, the untemplated nucleotide region includes a sequence that is at least partially complementary to the untemplated nucleotides added on to the 3′ end of the extended capture probe. In some embodiments, the untemplated nucleotide region includes a sequence that is 1, 2, 3, 4, 5, 6, 7, 8, 9, 10, or more nucleotides in length.

In some embodiments, the untemplated nucleotide region includes a series of G bases to complement the overhanging C bases at the 3′ end of a cDNA molecule. In some embodiments, the series of G bases can include 1 G base, 2 G bases, 3 G bases, 4 G bases, 5 G bases, or more than 5 G bases. In some embodiments, the hybridization region can include at least one base in addition to at least one G base. In other embodiments, the hybridization can include bases that are not a G base. In some embodiments, the template region and hybridization region are separated by a spacer. In some embodiments, the reverse complement of the TSO (rcTSO) is incorporated at the 3′ end of the cDNA molecule when the TSO binds to the untemplated nucleotides on the cDNA molecule and the reverse transcriptase reverse transcribes the TSO.

In some embodiments, the method includes contacting the analyte bound to the capture probe with a composition that includes a TSO primer. In some embodiments, the TSO primer includes a sequence that is at least partially complementary to the rcTSO sequence. In some embodiments, the TSO primer includes a sequence that is 1, 2, 3, 4, 5, 6, 7, 8, 9, 10, 11, 12, 13, 14, 15, 16, 17, 18, 19, 20, 21, 22, 23, 24, 25, or nucleotides in length. In some embodiments, the TSO primer includes any of the deoxyribonucleic acids, ribonucleic acids, modified nucleic acids, or any combination therein (e.g., any of the nucleotide derivatives or combinations thereof described herein). In some embodiments, the TSO primer includes RNA bases. In some embodiments, the TSO primer does not include RNA bases.

In some embodiments, the TSO primer is a single-stranded nucleic acid where the 3′ end is used as a chemical substrate for a nucleic acid polymerase in a nucleic acid extension reaction. In some embodiments, the TSO primer is used as a chemical substrate for a second strand synthesis where the extended capture probe is used as a template in a nucleic acid extension reaction, where the second strand is complementary to all or a portion of the cDNA molecule and all or a portion of the capture probe. In some embodiments, the TSO primer and a first enzyme (e.g., a reverse transcriptase with DNA polymerase functionality) are used in second strand synthesis, where the second strand synthesis occurs in the same reaction as the reverse transcription. In some embodiments, the TSO primer and a second enzyme (e.g., a DNA polymerase) are used in a second strand synthesis reaction, where the second strand synthesis occurs in the same reaction as the reverse transcription.

In some embodiments, the method includes contacting the analyte bound to the capture probe with a composition that includes a TSO blocking moiety. In some embodiments, the TSO blocking moiety is a nucleotide sequence that is 1, 2, 3, 4, 5, 6, 7, 8, 9, 10, 11, 12, 13, 14, 15, 16, 17, 18, 19, 20, 21, 22, 23, 24, 25, or nucleotides in length. In some embodiments, the TSO blocking moiety is a nucleotide sequence that is at least partially complementary to the TSO. In some embodiments, the TSO blocking moiety prohibits the TSO primer from interacting with the rcTSO. For example, the TSO blocking moiety can bind to the TSO primer thereby inhibiting the TSO primer from interacting the rcTSO. In some embodiment the TSO blocking moiety is a nucleotide sequence that is at least partially complementary to the rcTSO. In some embodiments, the TSO blocking moiety prohibits the rcTSO from interacting with the TSO primer. For example, the TSO blocking moiety can bind to the rcTSO thereby inhibiting the rcTSO from interacting with the TSO primer.

This disclosure features methods of improving sensitivity of spatial detection of an analyte in a biological sample where a second strand is circularized by denaturing the second strand under conditions wherein a first portion of the second strand and a second portion of the second strand de-hybridize from the extended capture probe and are hybridized to a splint oligonucleotide, ligated (e.g., circularized), and amplified.

In some embodiments, denaturing includes temperature modulation. For example, a first portion and a second portion have predetermined annealing temperatures based on the nucleotide composition (A, G, C, or T) within the known sequence. In some embodiments, the temperature is modulated up to 5° C., up to 10° C., up to 15° C., up to 20° C., up to 25° C., up to 30° C., or up to 35° C. above the predetermined annealing temperature. In some embodiments, the temperature is modulated at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35° C. above the predetermined annealing temperature. In some embodiments, once the temperature is modulated to a temperature above the predetermined annealing temperature, the temperature is cooled down to the predetermined annealing temperature at a ramp rate of about 0.1° C./second to about 1.0° C./second (e.g., about 0.1° C./second to about 0.9° C./second, about 0.1° C./second to about 0.8° C./second, about 0.1° C./second to about 0.7° C./second, about 0.1° C./second to about 0.6° C./second, about 0.1° C./second to about 0.5° C./second, about 0.1° C./second to about 0.4° C./second, about 0.1° C./second to about 0.3° C./second, about 0.1° C./second to about 0.2° C./second, about 0.2° C./second to about 1.0° C./second, about 0.2° C./second to about 0.9° C./second, about 0.2° C./second to about 0.8° C./second, about 0.2° C./second to about 0.7° C./second, about 0.2° C./second to about 0.6° C./second, about 0.2° C./second to about 0.5° C./second, about 0.2° C./second to about 0.4° C./second, about 0.2° C./second to about 0.3° C./second, about 0.3 to about 1.0° C./second, about 0.3° C./second to about 0.9° C./second, about 0.3° C./second to about 0.8° C./second, about 0.3° C./second to about 0.7° C./second, about 0.3° C./second to about 0.6° C./second, about 0.3° C./second to about 0.5° C./second, about 0.3° C./second to about 0.4° C./second, about 0.4° C./second to about 1.0° C./second, about 0.4° C./second to about 0.9° C./second, about 0.4° C./second to about 0.8° C./second, about 0.4° C./second to about 0.7° C./second, about 0.4° C./second to about 0.6° C./second, about 0.4° C./second to about 0.5° C./second, about 0.5° C./second to about 1.0° C./second, about 0.5° C./second to about 0.9° C./second, about 0.5° C./second to about 0.8° C./second, about 0.5° C./second to about 0.7° C./second, about 0.5° C./second to about 0.6° C./second, about 0.6° C./second to about 1.0° C./second, about 0.6° C./second to about 0.9° C./second, about 0.6° C./second to about 0.8° C./second, about 0.6° C./second to about 0.7° C./second, about 0.7° C./second to about 1.0° C./second, about 0.7° C./second to about 0.9° C./second, about 0.7° C./second to about 0.8° C./second, about 0.8° C./second to about 1.0° C./second, about 0.8° C./second to about 0.9° C./second, or about 0.9° C./second to about 1.0° C./second).

In some embodiments, denaturing includes temperature cycling. In some embodiments, denaturing includes alternating between denaturing conditions (e.g., a denaturing temperature) and non-denaturing conditions (e.g., annealing temperature).

(d) Digestion of the Amplified Second Stand and Amplification of the Fragments

This disclosure features methods of improving sensitivity of spatial detection of an analyte in a biological sample where the method includes digesting (or fragmenting) an amplified second strand to produce second strand fragments. In a non-limiting example, the digesting step includes inducing a plurality of double-stranded breaks in an amplified second strand. In some embodiments, the double-stranded break occurs at a specific nucleic acid position. In some embodiments, the second strand fragment includes a sequence of the second strand or a complement thereof and a sequence of the spatial barcode or a complement thereof.

In some embodiments, a double stranded break is induced using a restriction enzyme and a restriction site. In such cases because the product of an RCA reaction is a continuous single-stranded molecule (e.g., amplified second strand), a portion of the single-stranded molecule (e.g., amplified second strand) is made into a double stranded molecule. In some embodiments, the single-stranded molecule is converted into a double stranded molecule by contacting the single-stranded molecule with an oligonucleotide that is substantially complementary to a portion of the single-stranded molecule (e.g., amplified second strand) and includes a sequence that can serve as a restriction site for a restriction enzyme.

In some embodiments, the restriction site is not present in the sequence of the analyte bound to the capture probes. In such cases, when the amplified second strand is contacted with the oligonucleotide and the restriction enzyme, the oligonucleotide will not hybridize to the portion of the second strand that includes a sequence derived from the analyte. In some embodiments, the restriction site sequence is present in the second portion of the second strand.

In some embodiments, the digesting step includes contacting the amplified second strand with an oligonucleotide, where upon hybridization forms a double-stranded sequence with the amplified second strand. In some embodiments, the oligonucleotide includes one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides. In some embodiments, the oligonucleotide is substantially complementary to a portion of the amplified second strand. By substantially complementary, it is meant that the oligonucleotide is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in the amplified second strand. In some embodiments, following digestion, the residual oligonucleotide that remains hybridized. In some embodiments, following digestion, the oligonucleotide is de-hybridized from the amplified second strand.

In some embodiments, the oligonucleotide includes a 3′ blocking moiety. The 3′ blocking moiety prevents the oligonucleotide from being used as a primer in an RCA reaction. In some embodiments, the free 3′ end of the oligonucleotide can be blocked by chemical modification, e.g., addition of an azidomethyl group as a chemically reversible capping moiety such that the capture probes do not include a free 3′ end. Non-limiting examples of 3′ modifications include dideoxy C-3′ (3′-ddC), 3′ inverted dT, 3′ C3 spacer, 3′ Amino, and 3′ phosphorylation.

In some embodiments where the digesting step includes digesting the amplified second strand with a restriction enzyme, the restriction enzyme can induce a blunt end or sticky end (e.g., not a blunt end) break. In cases where digestion results in one or more blunt ends, the second strand fragments that include the blunt ends can be dephosphorylated (e.g., Shrimp Alkaline Phosphatase (SAP)) so that the ends do not reanneal or anneal to another blunt ended second strand fragment.

In some embodiments, the restriction enzyme is a rare-cutting restriction endonuclease. A rare-cutting restriction endonuclease includes a restriction site about once per 10 kilobases (kb), about once per 20 kb, about once per 30 kb, about once per 40 kb, once per 50 kb, about once per 60 kb, about one per 70 kb, about once per 80 kb, about once per 90 kb, about once per 100 kb, about once per 200 kb, about once per 300 kb, about once per 400 kb, about once per 500 kb, about once per 750 kb, or about once per 1 megabase (mb) of gDNA. In some embodiments, the restriction enzymes includes restriction enzymes with a 6-bp restriction site or an 8-bp restriction site. Non-limiting examples of restriction enzymes that can be used to digest the amplified second strand, include NotI, SalI, SfiI, NruI, MluI, SacII, and BssHII. In some embodiment, the restriction enzymes is NotI. In some embodiments, the restriction enzyme is SfiI. In some embodiments, the restriction enzyme can be a non-naturally occurring restriction enzyme.

Non-limiting examples of other methods for inducing a double-stranded DNA break include target specific nucleases (e.g., a CRISPR/Cas system where a Cas nuclease is directed to a specific sequence on the amplified second strand).

In some embodiments, the method further includes amplifying the second strand fragment, thereby generating an amplified second strand fragment(s). In some embodiments, amplifying the second strand fragments includes circle-to-circle amplification as described in Dahl et al. PNAS, 101(13): 4548-4553 (2004), the entire contents of which are incorporated herein by reference. In some embodiments, the method further includes determining the sequence of the amplified second strand fragment, wherein the determined sequence of the amplified second strand fragment includes the spatial barcode sequence of the capture probe or a complementary sequence thereof, and using the determined sequence of the amplified second strand fragment or the spatial barcode to spatially detect the analyte in the biological sample.

(e) Slides, Biological Samples, and Analytes

This disclosure features a method for identifying a location of an analyte in a biological sample using a substrate (e.g., a first substrate) that includes a plurality of capture probes, where a capture probe of the plurality of capture probes include a capture domain but no spatial barcode. In some embodiments, the capture probe is affixed to the substrate at a 5′ end. In some embodiments, the plurality of capture probes are uniformly distributed on a surface of the substrate. In some embodiments, the plurality of capture probes are located on a surface of the substrate but are not distributed on the substrate according to a pattern. In some embodiments, the substrate (e.g., a second substrate) includes a plurality of capture probes, where a capture probe of the plurality of capture probes includes a capture domain and a spatial barcode.

In some embodiments, the capture domain includes a sequence that is at least partially complementary to the analyte or the analyte derived molecule. In some embodiments, the capture domain of the capture probe includes a poly(T) sequence. In some embodiments, the capture domain includes a functional domain. In some embodiments, the functional domain includes a primer sequence. In some embodiments, the capture probe includes a cleavage domain. In some embodiments, the cleavage domain includes a cleavable linker from the group consisting of a photocleavable linker, a UV-cleavable linker, an enzyme-cleavable linker, or a pH-sensitive cleavable linker.

In some embodiments, the capture domain of the capture probe includes a non-homopolymeric sequence. In some embodiments, the capture domain is a defined sequence. In such cases, the defined sequence can be substantially complementary to a portion of the amplified padlock oligonucleotide. When the capture probe is substantially complementary to a portion of the amplified oligonucleotide, the capture domain can be used to capture all or a portion of the padlock oligonucleotide. In some instances, a defined sequence is about 5 to 50 nucleotides in length (e.g., about 5 to 25, about 5 to 20, about 10 to 25, about 10 to 20). In some instances, the length of the defined sequence is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides long. Defined sequences can include natural or synthetic nucleic acids. It is appreciated that a skilled artisan can design the defined sequence in order to capture a particular target or particular targets of interest.

In some embodiments, the capture domain of the capture probe includes a random sequence. In some embodiments, the capture domain includes a non-random sequence. For example, a capture domain with a non-random sequence can include, without limitation, a defined sequence or a homopolymeric sequence.

In some embodiments, the biological sample includes a FFPE sample. In some embodiments, the biological sample includes a tissue section. In some embodiments, the biological sample includes a fresh frozen sample. In some embodiments, the biological sample includes live cells.

The methods provided herein can be applied to analyte or analyte derived molecules including, without limitation, a second strand cDNA molecule (“second strand”). In some embodiments, the analyte or analyte derived molecules include RNA and/or DNA.

(f) Compositions and Kits

In some instances, disclosed herein are compositions and kits that are used to carry out the methods described herein. In some embodiments, the kit includes a splint oligonucleotide and a ligase (e.g., a T4 DNA ligase (Rnl2), a SplintR ligase, a single stranded DNA ligase, or a T4 DNA ligase). In some embodiments, the kit further includes one or more amplification primers (e.g., two or more primers, three or more primers, four or more primers, five or more primers, six or more primers, seven or more primers, eight or more primers, nine or more primers, or ten or more primers) and a polymerase (e.g., a Phi29 DNA polymerase). In some embodiments, the kit further includes an oligonucleotide and a restriction enzyme (e.g., any of the exemplary restriction enzymes described herein).

In some embodiments, the kit further includes a first substrate including a plurality of capture probes, wherein a capture probe of the plurality includes and a capture domain, wherein the analyte is capable of hybridizing to the capture domain. It is appreciated that the kit can include any of the elements of the substrate, array, or capture probes as described herein.

In some embodiments, a kit used to carry out the methods described herein includes: a substrate for spatial detection of an analyte; one or more splint oligonucleotides and a ligase; one or more RCA primers and a Phi29 DNA polymerase; and instructions for performing any of the methods described herein.

In some embodiments, a kit used to carry out the methods described herein includes: a substrate for spatial detection of an analyte; one or more splint oligonucleotides and a ligase; one or more RCA primers and a Phi29 DNA polymerase; one or more oligonucleotides and one or more restriction enzymes; and instructions for performing any of the methods described herein.

This disclosure features compositions including the circularized second strand, wherein the circularized second strand includes a first portion of the second strand, a portion of the second strand that remained hybridized to the extended capture probe, a second portion of the second strand, and a portion of the backbone sequence of the splint oligonucleotide.

### EXAMPLES

### Example 1—Method for Improving Sensitivity of Spatial Detection of an Analyte

This example provides an exemplary method for improving sensitivity of spatial detection of an analyte in a biological sample. In a non-limiting example, the method includes: hybridizing the analyte to a capture probe, creating a second strand that is complementary to a portion of the analyte and the extended capture probe, denaturing the second strand under conditions where a first portion of the second strand and a second portion of the second strand de-hybridize from the extended capture probe; hybridizing a splint oligonucleotide to the partially denatured second strand and using the split oligonucleotide to ligate the first and second portions together, thereby creating a circularized second strand; amplifying the circularized second strand, thereby creating an amplified second strand; and determining all or part of the sequence of the amplified second strand to spatially detect the analyte in the biological sample.

Briefly, a sample (e.g., an FFPE sample) is decrosslinked to remove formaldehyde crosslinks within the sample thereby releasing the analytes for spatial detection. The tissue samples are incubated with an HCl solution for 1 minute, repeated twice for a total of 3 minutes. Following HCl incubations, the tissue sections are incubated at 70° C. for 1 hour in TE pH 9.0. TE is removed and the tissues are incubated in 1×PBS-Tween for 15 minutes.

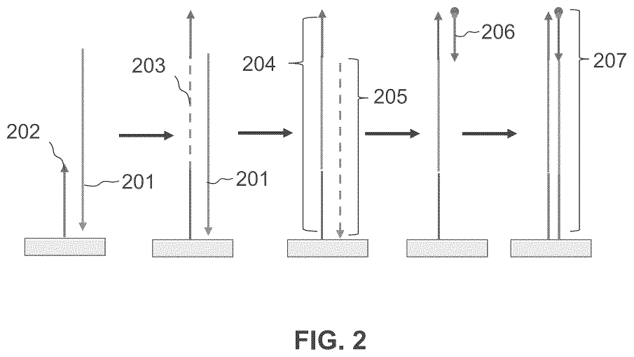

FIG. 2 shows exemplary, non-limiting, steps for second strand synthesis. The analyte 201 is hybridized to a capture probe 202 via a capture probe binding domain. A nucleic acid extension reaction (indicated by the dotted line and numeral 203) using a DNA polymerase is used to extend the capture probe thereby creating an extended capture probe 204. The analyte is removed (indicated by the dotted line and numeral 205) from the extended capture probe. A second strand synthesis primer 206 is hybridized to the extended capture probe. The extended capture probe is then copied using the second strand synthesis primer, thereby creating a second strand 207 that includes a sequence of the analyte or a complement thereof and the sequence of the spatial barcode or a complement thereof. The second strand remains hybridized to the extended capture probe.

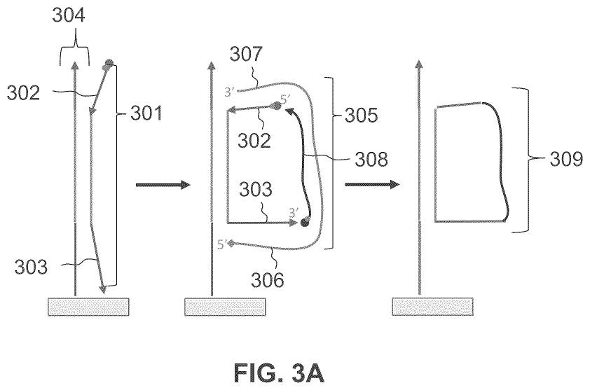

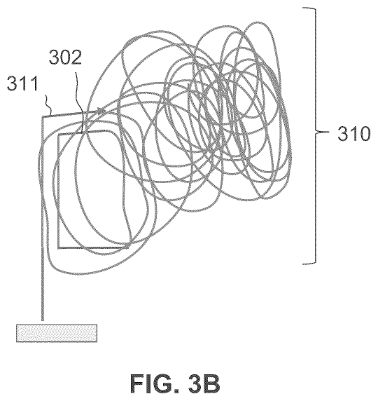

As show in FIG. 3A, the second strand 301 is denatured under conditions where a first portion 302 of the second strand and a second portion 303 of the second strand de-hybridize from the extended capture probe 304. Denaturing conditions include heating the second strand to 10° C. above the predetermined annealing temperature for 5 min and cooling at a rate of 0.5° C./second back down to the predetermined annealing temperature.

The splint oligonucleotide 305 includes a first sequence 306 that is substantially complementary to the second portion 303, and a second sequence 307 that is substantially complementary to the first portion 302. Following de-hybridizing of the first portion 302 and second portion 303, a splint oligonucleotide 305 is hybridized to the first portion 302 and the second portion 303.

The splint oligonucleotide mediates ligation between the second portion and the first portion, thereby creating a circularized second strand. Briefly, a nucleic acid extension reaction 308 is used to extend the second portion of the second strand in order to place the second portion directly adjacent to the first portion by using the middle section of the splint oligonucleotide as the template. The second portion is ligated to the first portion, thereby creating a circularized second strand 309.

As shown in FIG. 3B, the circularized second strand is amplified to create an amplified second strand 310. Amplification includes rolling circle amplification where the 3′ end of the extended capture probe 311 is hybridized to the circularized second strand (e.g., the first portion 302 of the second strand included in the circularized second strand). Finally, all or part of the sequence of the amplified second strand is sequenced and the sequence is used to determine the spatial location of the analyte in the biological sample.

### Example 2—Digestion of the Amplified Second Strand and Analysis of Second Strand Fragments

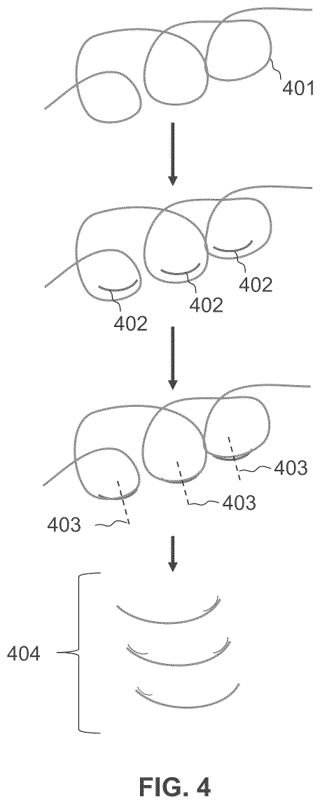

This example provides an exemplary method for improving sensitivity of spatial detection of an analyte in a biological sample. In a non-limiting example, the method includes digesting an amplified second strand thereby generating second strand fragments. As shown in FIG. 4, the amplified second strand 401 is contacted with an oligonucleotide 402 where the oligonucleotide includes a sequence that is substantially complementary to a portion of the amplified second strand. Hybridizing the oligonucleotide to the amplified second strand creates one or more double stranded restriction sites in the amplified second strand. The amplified second strand including the restriction site is contacted with a restriction enzyme that generates double-stranded breaks (as indicated by the dashed line and numeral 403). The double stranded breaks create restriction enzyme fragments of the amplified second strand, thereby creating second strand fragments 404. All or part of the sequence of the fragments of the amplified second strand (e.g., the second strand fragments) is determined and used to spatially detect the analyte in the biological sample.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A method of determining location and/or abundance of an analyte in a biological sample, the method comprising:
(a) hybridizing the analyte to a capture probe on an array, wherein the capture probe comprises a spatial barcode and a capture domain;
(b) extending the capture probe using the analyte as a template, thereby generating an extended capture probe, and generating a second strand comprising a sequence that is complementary to (i) the analyte or a complement thereof and (ii) the spatial barcode or a complement thereof;
(c) denaturing the second strand from the extended capture probe under conditions wherein a 5′ end of the second strand and a 3′ end of the second strand dehybridize from the extended capture probe;
(d) hybridizing a splint oligonucleotide both to the 5′ end of the second strand and to the 3′ end of the second strand;
(e) generating a circularized second strand;
(f) amplifying the circularized second strand, thereby creating an amplified second strand; and
(g) determining all or part of the sequence of the amplified second strand to determine the location and/or the abundance of the analyte in the biological sample.
